(19) United States
(12) Patent Application Publication
(10) Pub. No.: US 2011/0171064 A1
(43) Pub. Date: Jul. 14, 2011

(54) HYDROGEN PEROXIDE AND AMMONIA DECONTAMINATION OF A FOREIGN AGENT

(75) Inventor: E. William SARVER, Havre de Grace, MD (US)

(73) Assignee: Teledyne Brown Engineering, Inc., Huntsville, AL (US)

(21) Appl. No.: 12/759,439

(22) Filed: Apr. 13, 2010

Related U.S. Application Data
(60) Provisional application No. 61/174,364, filed on Apr. 30, 2009.

(51) Int. Cl. A61L 2/18

(52) U.S. Cl. 422/28; 422/292; 422/119

(57) ABSTRACT

A method for effectively decontaminating chemical agents to acceptable levels of contamination by applying hydrogen peroxide and ammonia decontaminants, and a mechanism for delivering the decontaminants.

### CROSS REFERENCE TO PRIOR APPLICATIONS

This application claims priority and the benefit thereof from a U.S. Provisional Application No. 61/174,364, filed on Apr. 30, 2009 and entitled HYDROGEN PEROXIDE AND AMMONIA DECONTAMINATION OF A FOREIGN AGENT, the entire contents of which are herein incorporated by reference.

### STATEMENT REGARDING FEDERALLY SPONSORED RESEARCH OR DEVELOPMENT

This invention was made with Government support under contract (W911SR-07-C-0092) awarded by the U.S. Army Research, Development, and Engineering Command (RDECOM) to Teledyne Brown Engineering, Inc. The Government has certain rights in the invention.

### FIELD OF THE INVENTION

The invention relates to a method for effectively decontaminating chemical agents to acceptable levels of contamination by applying hydrogen peroxide and ammonia decontaminants, and a mechanism for delivering the decontaminants.

### BACKGROUND OF THE INVENTION

S-[2-(diisopropylamino)ethyl]-O-ethyl methylphosphonothioate (VX), a commonly known nerve agent used in chemical warfare, is an extremely toxic substance. The decontamination of VX has plagued the military for many years. Previous attempts at decontaminating VX with the vaporous hydrogen peroxide (VHP)™ and modified vaporous hydrogen peroxide (mVHP)™ processes did not reduce the contaminant to acceptable levels of decontamination.

U.S. Patent Application Publication number 2008/0267819 A1 to Michael A. Bacik, et. al., discloses a portable decontamination unit that may employ a shipping container functioning as a decontamination chamber, and a decontamination process.

U.S. Patent Application Publication number 2008/0279722 A1 to Michael A. Bacik, et. al., discloses a portable decontamination unit that may be ruggedized for use in hostile environments, and a decontamination process.

Steris Corporation (<www.steris.com>) developed mVHP decontamination technology in cooperation with the United States Army as part of an ongoing effort to optimize VHP technology for use against chemical and biological warfare agents.

A need exists to decontaminate the VX to acceptable lower detectable limit (LDL) levels. The invention applies a process to decontaminate the foreign agent, and repeating the process at least one or more times until the VX is reduced to an acceptable level.

### SUMMARY OF THE INVENTION

The present invention provides an apparatus, a process and a computer program for successfully decontaminating chemical warfare agent VX by applying a multi-step process until the foreign agent is decontaminated to acceptable LDL levels.

According to an aspect of the invention, a method is provided for decontaminating a foreign agent, comprising: adding hydrogen peroxide to the foreign agent, and after the reaction slows, adding ammonia to the foreign agent, and after the reaction slows, removing the hydrogen peroxide and ammonia, and then repeating this sequence at least one time, but possibly several times, until the foreign agent is decontaminated to an acceptable level.

According to a further aspect of the invention, a decontamination apparatus is provided for decontaminating an article contaminated with the foreign agent. The decontamination apparatus comprises: a decontamination container built to receive and house the foreign agent; a hydrogen peroxide dispenser configured to dispense hydrogen peroxide to the container; and an ammonia dispenser configured to dispense ammonia to the container. The decontamination apparatus may comprise a hydrogen peroxide pump, a flash vaporizer and an air pump. The decontamination apparatus may be configured as a closed loop or open loop system. The container may comprise: an inlet and inlet valve configured to admit the hydrogen peroxide; an inlet and inlet valve configured to admit the ammonia; an outlet and outlet valve configured with a timer device to release the hydrogen peroxide and ammonia mixture from the container; and a monitoring and recording device configured to measure inlet and outlet flows, temperature, pH levels, relative humidity, hydrogen peroxide concentration, and composition inside the container. The hydrogen peroxide and ammonia dispensers may comprise a release valve and timer that regulates the flow of hydrogen peroxide and ammonia to the container, respectively. The article contaminated with the foreign agent may comprise at least one of an animal, a chemical, clothing, a tool, an object, or a device.

According to a still further aspect of the invention, a transportable decontamination apparatus is provided for decontaminating an article contaminated with the foreign agent. The transportable decontamination apparatus comprises: a portable decontamination container built to receive and house the foreign agent, two dispensers, and a monitoring and recording device; a transportable hydrogen peroxide dispenser residing within the container and configured to dispense hydrogen peroxide within the container; a transportable ammonia dispenser residing within the container and configured to dispense ammonia within the container; and a transportable monitoring and recording device that can be connected to the container and dispensers, and configured to measure inlet and outlet flows, temperature, pH levels, and composition inside the container. The decontamination apparatus may comprise a hydrogen peroxide pump, a flash vaporizer and an air pump. The decontamination apparatus may be configured as a closed loop or open loop system. The hydrogen peroxide and ammonia dispensers may contain a release valve and timer that regulates the flow of hydrogen peroxide and ammonia to the container, respectively. The article contaminated with the foreign agent may comprise at least one of an animal, a chemical, clothing, a tool, an object, or a device.

According to a still further aspect of the invention, a computer program is provided for decontaminating a foreign agent. The computer program may be embodied in a computer readable medium, the medium may include a plurality of instructions or code segments, which when executed on a general purpose computer cause: adding hydrogen peroxide to the foreign agent, and after the reaction slows, adding ammonia to the foreign agent and hydrogen peroxide, and after the reaction slows, removing the hydrogen peroxide and ammonia, and then repeating this sequence at least one time, but possibly several times, until the foreign agent is decontaminated to an acceptable level.

Additional features, advantages, and embodiments of the invention may be set forth or apparent from consideration of the detailed description and drawings. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and the various ways in which it may be practiced. In the drawings:

FIG. 1 shows an example of a flow diagram for a decontamination process, according to an embodiment of the invention;

FIG. 2 shows an example of a decontamination apparatus, according to an embodiment of the invention; and

FIG. 3 shows an alternative embodiment of the decontamination apparatus, according to an embodiment of the invention.

The present invention is further described in the detailed description that follows.

### DETAILED DESCRIPTION OF THE INVENTION

The embodiments of the invention and the various features and advantageous details thereof are explained more fully with reference to the non-limiting embodiments and examples that are described and/or illustrated in the accompanying drawings and detailed in the following attached description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention. Moreover, it is noted that like reference numerals represent similar parts throughout the several views of the drawings.

The present invention provides a process to decontaminate foreign agents, such as, e.g., VX, GD, derivatives of VX or GD, or the like, using an oxidant such as, e.g., hydrogen peroxide, followed by a pH level adjustment. The process may be repeated several times until the level of decontamination reaches a desired level, such as, for example, the level required by the United States Department of Defense's (DOD's) Joint Material Decontamination System (JMDS) Decontamination Program, less than about 0.3 mg per m2 for VX.

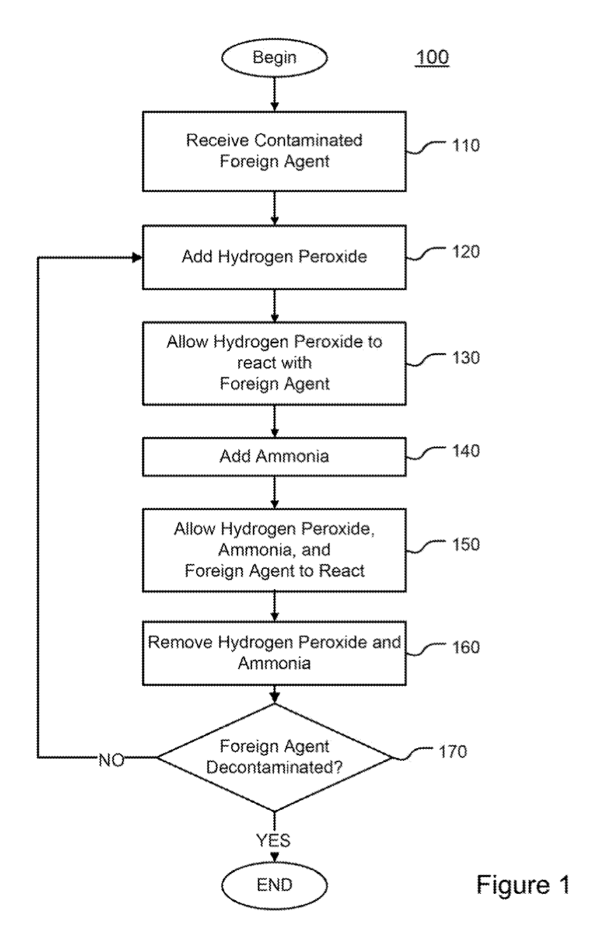

FIG. 1 shows an example of a flow diagram for a decontamination process 100, according to an aspect of the invention.

Referring to FIG. 1, the decontamination process may begin when a contaminated foreign agent (such as, e.g., VX, GD, or the like) is received, such as, e.g., when an article contaminated with the foreign agent is placed into a decontamination container (Step 110). Hydrogen peroxide may be added to the foreign agent (Step 120). For example, the hydrogen peroxide may be added by injecting the hydrogen peroxide (in gaseous or liquid form) into the decontamination chamber. The hydrogen peroxide may then be allowed to react with the foreign agent, until, for example, the reaction between the hydrogen peroxide and foreign agent nears its end (Step 130). In this regard, the amount of foreign agent may be reduced by, for example, about 80% of the initial, total amount of foreign agent. When the reaction between the hydrogen peroxide and foreign agent nears its end, ammonia may be added to the hydrogen peroxide and foreign agent (the reaction volume) to cause the reaction between the hydrogen peroxide and foreign agent to continue by, for example, neutralizing an acid by-product of the reaction (e.g., ethyl methylphophonic acid, or the like) (Step 140). The ammonia, hydrogen peroxide and foreign agent may be allowed to react until, for example, the reaction nears completion (such as, but not limited to, e.g., 99.9% completion) (Step 150).

After the reaction of the hydrogen peroxide, foreign agent and ammonia nears completion, hydrogen peroxide and ammonia may be removed (Step 160). For example, the hydrogen peroxide and ammonia may be removed from the decontamination container by, for example, opening a release valve in the decontamination container to allow the hydrogen peroxide and ammonia to be released through the valve, and/or activating a suction pump to remove the hydrogen peroxide and ammonia from the container while injecting, e.g., air into the container, to maintain a predetermined pressure within the container (such as, e.g., atmospheric pressure, or the like). A determination may be made to determine whether the foreign agent has been decontaminated to an acceptable level (such as, e.g., less than about 0.3 mg per m² for VX.) (Step 170). If it is determined that the foreign agent has not been decontaminated to an acceptable level (NO at Step 170), then hydrogen peroxide is added to the foreign agent (Step 120) and the process repeats, otherwise the process ends (YES at Step 170).

The hydrogen peroxide may comprise condensed hydrogen peroxide, non-condensed hydrogen peroxide, a mixture of condensed and non-condensed hydrogen peroxide, or the like. The hydrogen peroxide may be in a gaseous state, a liquid state, a solid state, or any combination of thereof.

The ammonia may comprise an alkaline gas, ammonium perhydroxide (NH₄OOH), or both. Further, the ammonia may be in a gaseous state, a liquid state, a solid state, or any combination of thereof.

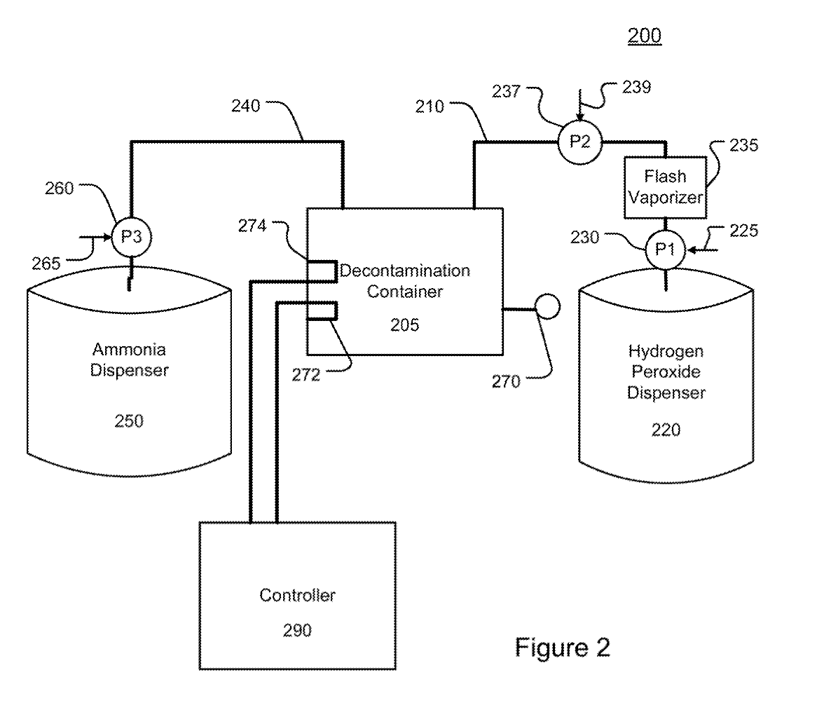

FIG. 2 shows an example of a decontamination apparatus 200 according to an embodiment of the invention. The decontamination apparatus 200 includes a decontamination container 205 to host (or contain) the foreign agent, a hydrogen peroxide dispenser 220 configured to dispense hydrogen peroxide to the container 205, a hydrogen peroxide pump (P1) 230, a flash vaporizer 235, an air pump (P2) 237, an ammonia dispenser 250 configured to dispense ammonia to the container 205, an ammonia pump (P3) 260, an outlet valve 270, a plurality of sensors 272, 274, and a controller 290.

Referring to FIG. 2, the container 205 may include a decontamination chamber (not shown) configured to receive and house the foreign agent, or an article containing the foreign agent. For example, the decontamination chamber may comprise an internal volume, weight, height, depth, and width appropriate to house articles contaminated with the foreign agent. The chamber may be constructed of a material appropriate to house the foreign agent, and it may be maintained at temperatures conducive to facilitating the chemical reactions. The chamber may comprise at least one door or other opening of suitable size to receive the article containing the foreign agent. The articles to be decontaminated may include an animal, a chemical, a tool, a device, an object, articles of clothing, or the like, without limitation.

The decontamination container 205 may be coupled to a first inlet conduit 210, which may be configured to supply the hydrogen peroxide from the hydrogen peroxide dispenser 220 into the decontamination chamber. In this regard, the hydrogen peroxide may be pumped from the hydrogen peroxide dispenser by means of the pump 230 and supplied to the flash vaporizer 235, where the hydrogen peroxide may be heated to a vapor form. The vapor hydrogen peroxide may then be pumped into the decontamination chamber by means of the air pump 237. The air pump 237 may be supplied with an external source of air in an open loop configuration. Alternatively, the air pump 237 may be supplied with air from the decontamination chamber in a closed loop configuration. The hydrogen peroxide may be provided from the hydrogen peroxide dispenser 220 at atmospheric pressure, or under pressure (such as, e.g., at pressures above (or below) atmospheric pressure). The pump 230, flash vaporizer 235 and/or air pump 237 may be located proximate the hydrogen peroxide dispenser 220 or the container 205. The air pump 237 and flash vaporizer 235 may be configured as a single unit (not shown). The pump 230 and/or air pump 237 may be configured to controllably supply the hydrogen peroxide to flow through the first inlet conduit 210 into the decontamination chamber. In this regard the, the pump 230 and/or air pump 237 may be manually or automatically activated. In the latter instance, the decontamination apparatus 200 may be provided with a timer (not shown) located on or near the hydrogen peroxide dispenser 220 to regulate the flow of hydrogen peroxide from the dispenser 220 at a predetermined rate. Alternatively (or additionally), the pump 230 and/or air pump 237 may be, for example, electrically actuated by the controller 290 via control lines 225, 239, respectively, to controllably supply and regulate the flow of hydrogen peroxide from the dispenser 220 at a predetermined rate.

The decontamination container 205 may be coupled to a second inlet conduit 240, which may be configured to supply ammonia from the ammonia dispenser 250 into the decontamination chamber. The ammonia may be supplied from the ammonia dispenser 250 at atmospheric pressure, or under pressure (such as, e.g., at pressures above (or below) atmospheric pressure), through a pump 260. The pump 260 may be configured to controllably supply the ammonia to flow through the inlet conduit 240 into the decontamination chamber. In this regard the, the pump 260 may be manually or automatically activated. In the latter instance, the decontamination apparatus 200 may be provided with a timer (not shown) located on or near the ammonia dispenser to supply and regulate the flow of ammonia from the dispenser 250 at a predetermined rate. Alternatively (or additionally), the pump 260 may be, for example, electrically actuated by the controller 290 via a control line 265 to controllably supply and regulate the flow of hydrogen peroxide from the dispenser 220 at a predetermined rate.

The controller 290 may include, but is not limited to, for example, an electronic device configured to accept data, perform prescribed mathematical and logical operations at high speed, and output the results of these operations. The controller 290 may further include, but is not limited to, for example, a personal computer, a laptop computer, a palmtop computer, a notebook computer, a desktop computer, a workstation, or the like. The controller 290 may be configured to receive sensor data from the plurality of sensors 272, 274, such as, but not limited to, for example, temperature data, pH data, relative humidity, hydrogen peroxide concentration, VX concentration data, and the like.

The controller 290 may also be configured to generate hydrogen peroxide injection control signals, ammonia injection control signals, hydrogen peroxide and ammonia release control signals, and the like. The hydrogen peroxide control signals may be supplied to the pump 230 (and/or flash vaporizer 235, air pump 237) to allow hydrogen peroxide to be added to the foreign agent in the decontamination chamber. The ammonia control signals may be supplied to the pump 260 to add ammonia to the foreign agent in the decontamination chamber. The hydrogen peroxide and ammonia release control signals may be supplied to the outlet valve 270 to allow the hydrogen peroxide and ammonia to be removed from the decontamination chamber.

The hydrogen peroxide dispenser 220 may comprise an internal volume, weight, height, depth, and width appropriate to house enough hydrogen peroxide to perform multiple iterations of the process, as necessary. The ammonia dispenser 250 may comprise an internal volume, weight, height, depth, and width appropriate to house enough ammonia to perform multiple iterations of the process, as necessary. The hydrogen peroxide dispenser and ammonia dispenser are constructed of appropriate material to house, respectively, the hydrogen peroxide and ammonia in the form it is dispensed.

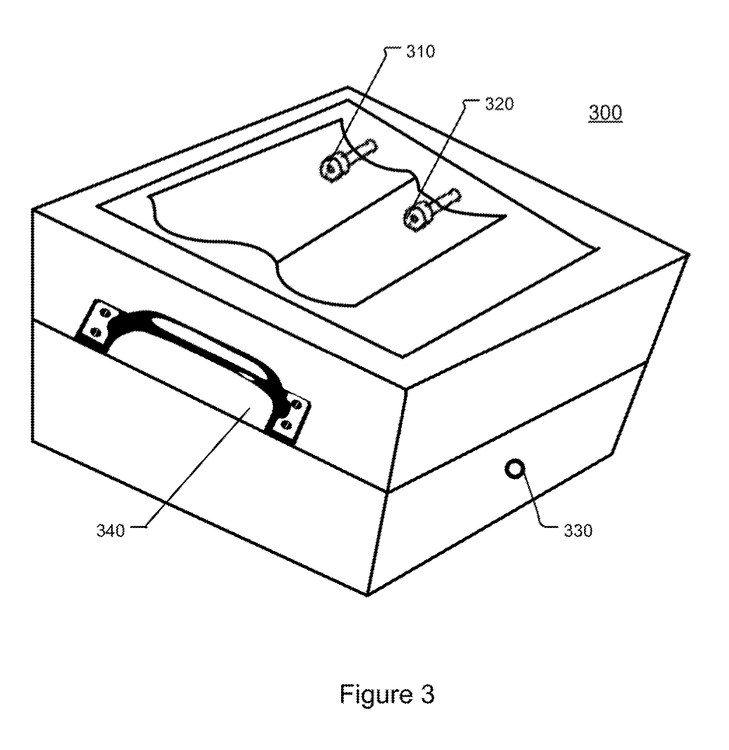

FIG. 3 shows an alternative embodiment of the decontamination apparatus, according to an embodiment of the invention.

Referring to FIG. 3, the portable decontamination apparatus 300 may contain the same configuration as the decontamination apparatus 200 in FIG. 2, except in portable form. A portable hydrogen peroxide dispenser (not shown) may be configured to reside in and connect to the portable decontamination apparatus at the first inlet valve 310, which connects to a hydrogen peroxide pump (not shown), a flash vaporizer (not shown) and an air pump (not shown). A portable ammonia dispenser (not shown) may be configured to reside in and connect to the portable decontamination apparatus at second inlet valve 320, which connects to an ammonia pump (not shown). A portable controller (not shown), with the same functional capability as the controller 290 in FIG. 2, may be configured to be transported in the portable decontamination apparatus.

The portable decontamination apparatus 300 may include a decontamination chamber (not shown) configured to receive and house the foreign agent, or an article containing the foreign agent. For example, the decontamination chamber may comprise an internal volume, weight, height, depth, and width appropriate to house articles contaminated with the foreign agent. The chamber may be constructed of a material appropriate to house the foreign agent, and it may be maintained at temperatures conducive to facilitating the chemical reactions. The chamber may comprise at least one door or other opening of suitable size to receive the article containing the foreign agent. The articles to be decontaminated may include an animal, a chemical, a tool, a device, an object, articles of clothing, or the like, without limitation.

The controller (not shown) may also be configured to generate hydrogen peroxide injection control signals, ammonia injection control signals, hydrogen peroxide and ammonia release control signals, and the like. The hydrogen peroxide control signals may be supplied to the hydrogen peroxide pump (not shown) to supply hydrogen peroxide to be added to the foreign agent in the decontamination chamber. The ammonia control signals may be supplied to the ammonia pump (not shown) to supply ammonia to be added to the foreign agent in the decontamination chamber. The hydrogen peroxide and ammonia release control signals may be supplied to the outlet valve 330 to allow the hydrogen peroxide and ammonia to be removed from the decontamination chamber.

The decontamination chamber may comprise straps (not shown) to hold the hydrogen peroxide and ammonia dispensers in place. At least one exhaust valve 330 located on the exterior of the portable unit may allow the removal of the hydrogen peroxide and ammonia from the portable unit. The portable decontamination apparatus may comprise at least one handle 340 on the exterior of the portable decontamination apparatus made of appropriate materials and dimensions for the portable decontamination apparatus to be carried. The portable decontamination apparatus may comprise at least two wheels (not shown) on the exterior of the unit of appropriate materials and dimensions for the unit for easy transport along a surface.

The controller 290 (shown in FIG. 2) may be provided with a computer program for decontaminating a foreign agent. The computer program may be embodied in a computer readable medium, the medium may include a plurality of instructions or code segments, which when executed on a general purpose computer cause each of the steps 110 through 170 (shown in FIG. 1) to be carried out in the decontamination apparatus 200 (shown in FIG. 2) or portable decontamination apparatus (shown in FIG. 3). For example, the controller 290, when executing the plurality of instructions or code segments, may cause hydrogen peroxide to be added to a foreign agent. After the reaction between the hydrogen peroxide and foreign agent slows, ammonia may be added to the foreign agent and hydrogen peroxide, and after the reaction slows, the hydrogen peroxide and ammonia may be removed. Then, this sequence of steps may be repeated at least one time, but possibly several times, until the foreign agent is decontaminated to an acceptable level.

By way of example, but not of limitation, an example of the present invention will now be given.

### Example 1

Twenty four two inch diameter glass coupons were placed into six separate Petri dishes (four coupons in each Petri dish). To nineteen of the coupons, about 0.51 mg of VX was deposited at the center of the coupons and allowed to spread across the surface of the coupons and the remaining five coupons were left uncontaminated as controls. The Petri dishes were allowed to age for one hour and then loaded into the decontamination chamber. The approximately 0.3 m³ chamber was charged with about 15 ml of liquid 35% hydrogen peroxide vaporized into it. The hydrogen peroxide was allowed to form a micro-condensate covering the surfaces of the chamber and coupons and to react for thirty minutes. At the end of the thirty minute reaction time, a one minute release into the chamber of about 1.6 liters of anhydrous ammonia gas and this mixture of hydrogen peroxide and ammonia was allowed to react for ten minutes. At the end of the reaction time the reacting gases of hydrogen peroxide and ammonia were allowed to escape and this step took ten minutes. This process was repeated an additional time and at the end of the second cycle during the venting process, two Petri dishes and their eight coupons were removed and analyzed for VX contact hazard. This process was repeated for a third and then a fourth cycle and as with the second cycle coupons were removed at the end of each and the coupons were removed and analyzed. The sampling times were 75, 115 and 155 minutes.

The samples were analyzed to measure surface contamination of VX. Table 1 below shows the mass recovered and percent recovered by sample number and location.

TABLE 1
ANALYTICAL RESULTS FROM A VX RUN
VX Run

| | | |
|---|---|---|
| LDL | 0.00008 mg | (0.158 mg/m2) |
| Challenge Mass: | 0.495 mg | P/F: 0.306 mg/m2 or 0.000155 mg |

Contact Hazard 1

| | | | | | Resulting | |
|---|---|---|---|---|---|---|
| | | Exposure | Mass | | Agent | |
| Position | Sample | Time | Recovered | % | Density | % |
| Code | ID | (min) | (mg) | Recovered | (mg/m2) | KPP |
| A1 | 573 | 75 | LDL | N/A | N/A | N/A |
| A2 | 574 | 75 | LDL | N/A | N/A | N/A |
| A3 | 575 | 75 | 0.000109 | 0.0% | 0.21 | 70% |
| A4 | 576 | 75 | 0.000097 | 0.0% | 0.19 | 63% |
| B5 | 577 | 75 | LDL | N/A | N/A | N/A |
| B6 | 578 | 75 | LDL | N/A | N/A | N/A |
| B7 | 579 | 75 | 0.000081 | 0.0% | 0.16 | 53% |
| B8 | 580B | 75 | LDL | N/A | N/A | N/A |
| C9 | 581 | 115 | LDL | N/A | N/A | N/A |
| C10 | 582 | 115 | LDL | N/A | N/A | N/A |
| C11 | 583 | 115 | LDL | N/A | N/A | N/A |
| C12 | 584 | 115 | LDL | N/A | N/A | N/A |
| D13 | 585B | 115 | LDL | N/A | N/A | N/A |
| D14 | 586B | 115 | LDL | N/A | N/A | N/A |
| D15 | 587B | 115 | LDL | N/A | N/A | N/A |
| D16 | 588B | 115 | LDL | N/A | N/A | N/A |
| E17 | 589 | 180 | LDL | N/A | N/A | N/A |
| E18 | 590 | 180 | LDL | N/A | N/A | N/A |
| E19 | 591 | 180 | LDL | N/A | N/A | N/A |
| E20 | 592 | 180 | LDL | N/A | N/A | N/A |
| F21 | 593 | 180 | LDL | N/A | N/A | N/A |
| F22 | 594 | 180 | LDL | N/A | N/A | N/A |
| F23 | 595 | 180 | LDL | N/A | N/A | N/A |
| F24 | 596 | 180 | LDL | N/A | N/A | N/A |

The results indicate that the threshold level of surface contamination after application of the multi-reaction step process was in the lower detectable limit (LDL) of less than about 0.3 mg per m² for VX of surface contamination, as compared with a starting concentration of 1 g/m² of surface contamination. This process was able to achieve the desired level of decontamination.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure.

What is claimed:

1. A method for decontaminating a foreign agent, comprising:
adding hydrogen peroxide to the foreign agent;
allowing the hydrogen peroxide to react with the foreign agent; and
adding ammonia to the foreign agent and hydrogen peroxide.

2. The method of claim 1, further comprising:
allowing the hydrogen peroxide and ammonia to react.

3. The method of claim 1, wherein the foreign agent is placed in a container, the method further comprising:
releasing the hydrogen peroxide and ammonia until both the hydrogen peroxide and ammonia are released from the container.

4. The method of claim 1, further comprising repeating the method of claim 1 until the foreign agent is decontaminated to an acceptable level.

5. The method of claim 1, wherein the hydrogen peroxide comprises condensed hydrogen peroxide.

6. The method of claim 1, wherein the hydrogen peroxide comprises non-condensed hydrogen peroxide.

7. The method of claim 1, wherein the ammonia comprises an alkaline gas.

8. The method of claim 1, wherein the ammonia comprises ammonium perhydroxide.

9. The method of claim 3, wherein the ammonia released from the container comprises ammonium perhydroxide.

10. A decontamination apparatus for decontaminating a foreign agent, comprising:
a container configured to receive the foreign agent;
a hydrogen peroxide dispenser configured to dispense hydrogen peroxide to the container; and
an ammonia dispenser configured to dispense ammonia to the container.

11. The apparatus according to claim 10, wherein the container comprises:
a decontamination chamber that is configured to receive and house the foreign agent;
a first inlet configured to admit the hydrogen peroxide into the decontamination chamber; and
a second inlet configured to admit the ammonia into the decontamination chamber.

12. The apparatus according to claim 11, further comprising:
a first pump configured to controllably supply the hydrogen peroxide to flow through the first inlet; and
a second pump configured to controllably supply the ammonia to flow through the second inlet.

13. The apparatus according to claim 11, wherein the container comprises an internal volume, weight, height, depth, and width appropriate to house an article contaminated with the foreign agent.

14. The apparatus according to claim 10, further comprising:
a flash vaporizer for vaporizing the hydrogen peroxide; and
an outlet configured to release a hydrogen peroxide and ammonia mixture.

15. The apparatus according to claim 14, wherein the outlet comprises at least one of a release valve and a timer device configured to release the hydrogen peroxide and ammonia mixture.

16. The apparatus according to claim 14, further comprising:
a inlet meter configured to measure inlet flows to the container; and
an outlet meter configured to measure outlet flows from the container.

17. The apparatus according claim 16, further comprising:
a recorder configured to record the inlet and outlet flows.

18: The apparatus according to claim 17, wherein the inlet meter or outlet meter are configured to measure at least one of a temperature, a pH level, and a content inside the container.

19. The apparatus according to claim 10, wherein the hydrogen peroxide dispenser comprises a release valve and timer that regulate the flow of hydrogen peroxide to the container.

20. The apparatus according to claim 10, wherein the ammonia dispenser comprises a release valve and timer that regulates the flow of ammonia to the container.

21. The apparatus according to claim 13, wherein the article comprises at least one of:
an animal;
a chemical;
a tool;
a device;
an object; and
clothing.
